SEQUENCE LISTING (16 sequences)

SEQ ID NO: 1 - DNA, 30 bp, Artificial Sequence
  Amplification Primer
gccgaatccatgagtgctcctaaactcctc

SEQ ID NO: 2 - DNA, 63 bp, Artificial Sequence
  Amplification primer
cgtcatgcagactcgggggaactgccctccgccaccctcaggtacactaaactcccgacttgg

SEQ ID NO: 3 - DNA, 63 bp, Artificial Sequence
  Amplification primer
ccaagtcgggagtttagtgtacctgagggtggcggagggcagttcccccgagtctgcatgacg

SEQ ID NO: 4 - DNA, 60 bp, Artificial Sequence
  Amplification primer
ggagacacaggctctagggaaatgtccacccccgccagttgtgggccaacctggagtttc

SEQ ID NO: 5 - DNA, 60 bp, Artificial Sequence
  Amplification primer
gaaactccaggttggcccacaactggcgggggtggacatttccctagagcctgtgtctcc

SEQ ID NO: 6 - DNA, 36 bp, Artificial Sequence
  Amplification primer
gcggcgctcgagctatgaccagatccgactcgcttg

SEQ ID NO: 7 - DNA, 4176 bp, Artificial Sequence
  Tri-hybrid antigen encoding sequence
atgagtgctcctaaactcctctctctgggctgtatcttcttccccttgctactttttcagcaggcccgggctcaattcccaagacagtgtgccactgttgaggctttgagaagtggtatgtgttgcccagacctgtcccctgtgtctgggcctgggacagaccgctgtggctcatcatcagggaggggcagatgtgaggcagtgactgcagactcccggccccacagccctcagtatccccatgatggcagagatgatcgggaggtctggcccttgcgcttcttcaataggacatgtcactgcaacggcaatttctcaggacacaactgtgggacgtgccgtcctggctggagaggagctgcctgtgaccagagggttctcatagtcaggagaaatcttctggacttaagtaaagaagaaaagaaccactttgtccgggccctggatatggcaaagcgcacaactcaccctttatttgtcattgccaccaggagatcagaagaaatactggggccagatggcaacacgccacaatttgagaacatttccatttataactactttgtttggacacactattactcagtcaaaaagactttccttggggtaggacaggaaagctttggtgaagtggatttctctcatgagggaccagcttttctcacatggcacaggtaccacctcctgcgtctggagaaagacatgcaggaaatgttgcaagagccttctttctcccttccttactggaattttgcaacggggaaaaatgtctgtgatatctgcacggatgacttgatgggatccagaagcaactttgattccactctaataagcccaaactctgtcttttctcaatggcgagtggtctgtgactccttggaagattatgataccctgggaacactttgtaacagcaccgaggatgggccaattaggagaaatccagctggaaatgtggccagaccaatggtgcaacgtcttcctgaaccacaggatgtcgctcagtgcttggaagttggtttatttgacacgcctcctttttattccaactctacaaacagtttccgaaacacagtggaaggttacagtgaccccacgggaaagtatgaccctgctgttcgaagtcttcacaatttggctcatctattcctgaatggaacagggggacaaacccatttgtctccaaatgatcctatttttgtcctcctgcacaccttcacagatgcagtctttgatgaatggctgaggagatacaatgctgatatatccacatttccattggaaaatgcccctattggacataatagacaatacaacatggtgccattctggcccccagtcaccaacacagaaatgtttgttactgctccagacaacctgggatacacttatgaaattcaatggccaagtcgggagtttagtgtacctgagggtggcggagggcagttcccccgagtctgcatgacggtggacagcctagtgaacaaggagtgctgcccacgcctgggtgcagagtcggccaatgtctgtggctctcagcaaggccgggggcagtgcacagaggtgcgagccgacacaaggccctggagtggtccctacatcctacgaaaccaggatgaccgtgagctgtggccaagaaaattcttccaccggacctgcaagtgcacaggaaactttgccggctataattgtggagactgcaagtttggctggaccggtcccaactgcgagcggaagaaaccaccagtgattcggcagaacatccattccttgagtcctcaggaaagagagcagttcttgggcgccttagatctcgcgaagaagagagtacaccccgactacgtgatcaccacacaacactggctgggcctgcttgggcccaatggaacccagccgcagtttgccaactgcagtgtttatgatttttttgtgtggctccattattattctgttagagatacattattaggaccaggacgcccctacagggccatagatttctcacatcaaggacctgcatttgttacctggcaccggtaccatttgttgtgtctggaaagagatctccagcgactcattggcaatgagtcttttgctttgccctactggaactttgccactgggaggaacgagtgtgatgtgtgtacagaccagctgtttggggcagcgagaccagacgatccgactctgattagtcggaactcaagattctccagctgggaaactgtctgtgatagcttggatgactacaaccacctggtcaccttgtgcaatggaacctatgaaggtttgctgagaagaaatcaaatgggaagaaacagcatgaaattgccaaccttaaaagacatacgagattgcctgtctctccagaagtttgacaatcctcccttcttccagaactctaccttcagtttcaggaatgctttggaagggtttgataaagcagatgggactctggattctcaagtgatgagccttcataatttggttcattccttcctgaacgggacaaacgctttgccacattcagccgccaatgatcccatttttgtggttcttcattcctttactgatgccatctttgatgagtggatgaaaagatttaatcctcctgcagatgcctggcctcaggagctggcccctattggtcacaatcggatgtacaacatggttcctttcttccctccagtgactaatgaagaactctttttaacctcagaccaacttggctacagctatgccatcgatctgccagtttcagttgaagaaactccaggttggcccacaactggcgggggtggacatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtca

SEQ ID NO: 8 - protein, 1392 aa, Artificial Sequence [sequence omitted]

SEQ ID NO: 9 - DNA, 4104 bp, Artificial Sequence
  Tri-hybrid antigen coding sequence
caattcccaagacagtgtgccactgttgaggctttgagaagtggtatgtgttgcccagacctgtcccctgtgtctgggcctgggacagaccgctgtggctcatcatcagggaggggcagatgtgaggcagtgactgcagactcccggccccacagccctcagtatccccatgatggcagagatgatcgggaggtctggcccttgcgcttcttcaataggacatgtcactgcaacggcaatttctcaggacacaactgtgggacgtgccgtcctggctggagaggagctgcctgtgaccagagggttctcatagtcaggagaaatcttctggacttaagtaaagaagaaaagaaccactttgtccgggccctggatatggcaaagcgcacaactcaccctttatttgtcattgccaccaggagatcagaagaaatactggggccagatggcaacacgccacaatttgagaacatttccatttataactactttgtttggacacactattactcagtcaaaaagactttccttggggtaggacaggaaagctttggtgaagtggatttctctcatgagggaccagcttttctcacatggcacaggtaccacctcctgcgtctggagaaagacatgcaggaaatgttgcaagagccttctttctcccttccttactggaattttgcaacggggaaaaatgtctgtgatatctgcacggatgacttgatgggatccagaagcaactttgattccactctaataagcccaaactctgtcttttctcaatggcgagtggtctgtgactccttggaagattatgataccctgggaacactttgtaacagcaccgaggatgggccaattaggagaaatccagctggaaatgtggccagaccaatggtgcaacgtcttcctgaaccacaggatgtcgctcagtgcttggaagttggtttatttgacacgcctcctttttattccaactctacaaacagtttccgaaacacagtggaaggttacagtgaccccacgggaaagtatgaccctgctgttcgaagtcttcacaatttggctcatctattcctgaatggaacagggggacaaacccatttgtctccaaatgatcctatttttgtcctcctgcacaccttcacagatgcagtctttgatgaatggctgaggagatacaatgctgatatatccacatttccattggaaaatgcccctattggacataatagacaatacaacatggtgccattctggcccccagtcaccaacacagaaatgtttgttactgctccagacaacctgggatacacttatgaaattcaatggccaagtcgggagtttagtgtacctgagggtggcggagggcagttcccccgagtctgcatgacggtggacagcctagtgaacaaggagtgctgcccacgcctgggtgcagagtcggccaatgtctgtggctctcagcaaggccgggggcagtgcacagaggtgcgagccgacacaaggccctggagtggtccctacatcctacgaaaccaggatgaccgtgagctgtggccaagaaaattcttccaccggacctgcaagtgcacaggaaactttgccggctataattgtggagactgcaagtttggctggaccggtcccaactgcgagcggaagaaaccaccagtgattcggcagaacatccattccttgagtcctcaggaaagagagcagttcttgggcgccttagatctcgcgaagaagagagtacaccccgactacgtgatcaccacacaacactggctgggcctgcttgggcccaatggaacccagccgcagtttgccaactgcagtgtttatgatttttttgtgtggctccattattattctgttagagatacattattaggaccaggacgcccctacagggccatagatttctcacatcaaggacctgcatttgttacctggcaccggtaccatttgttgtgtctggaaagagatctccagcgactcattggcaatgagtcttttgctttgccctactggaactttgccactgggaggaacgagtgtgatgtgtgtacagaccagctgtttggggcagcgagaccagacgatccgactctgattagtcggaactcaagattctccagctgggaaactgtctgtgatagcttggatgactacaaccacctggtcaccttgtgcaatggaacctatgaaggtttgctgagaagaaatcaaatgggaagaaacagcatgaaattgccaaccttaaaagacatacgagattgcctgtctctccagaagtttgacaatcctcccttcttccagaactctaccttcagtttcaggaatgctttggaagggtttgataaagcagatgggactctggattctcaagtgatgagccttcataatttggttcattccttcctgaacgggacaaacgctttgccacattcagccgccaatgatcccatttttgtggttcttcattcctttactgatgccatctttgatgagtggatgaaaagatttaatcctcctgcagatgcctggcctcaggagctggcccctattggtcacaatcggatgtacaacatggttcctttcttccctccagtgactaatgaagaactctttttaacctcagaccaacttggctacagctatgccatcgatctgccagtttcagttgaagaaactccaggttggcccacaactggcgggggtggacatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtca

SEQ ID NO: 10 - protein, 1368 aa, Artificial Sequence
  Tri-hybrid antigen
QFPRQCATVEALRSGMCCPDLSPVSGPGTDRCGSSSGRGRCEAVTADSRPHSPQYPHDGRDDREVWPLRFFNRTCHCNGNFSGHNCGTCRPGWRGAACDQRVLIVRRNLLDLSKEEKNHFVRALDMAKRTTHPLFVIATRRSEEILGPDGNTPQFENISIYNYFVWTHYYSVKKTFLGVGQESFGEVDFSHEGPAFLTWHRYHLLRLEKDMQEMLQEPSFSLPYWNFATGKNVCDICTDDLMGSRSNFDSTLISPNSVFSQWRVVCDSLEDYDTLGTLCNSTEDGPIRRNPAGNVARPMVQRLPEPQDVAQCLEVGLFDTPPFYSNSTNSFRNTVEGYSDPTGKYDPAVRSLHNLAHLFLNGTGGQTHLSPNDPIFVLLHTFTDAVFDEWLRRYNADISTFPLENAPIGHNRQYNMVPFWPPVTNTEMFVTAPDNLGYTYEIQWPSREFSVPEGGGGQFPRVCMTVDSLVNKECCPRLGAESANVCGSQQGRGQCTEVRADTRPWSGPYILRNQDDRELWPRKFFHRTCKCTGNFAGYNCGDCKFGWTGPNCERKKPPVIRQNIHSLSPQEREQFLGALDLAKKRVHPDYVITTQHWLGLLGPNGTQPQFANCSVYDFFVWLHYYSVRDTLLGPGRPYRAIDFSHQGPAFVTWHRYHLLCLERDLQRLIGNESFALPYWNFATGRNECDVCTDQLFGAARPDDPTLISRNSRFSSWETVCDSLDDYNHLVTLCNGTYEGLLRRNQMGRNSMKLPTLKDIRDCLSLQKFDNPPFFQNSTFSFRNALEGFDKADGTLDSQVMSLHNLVHSFLNGTNALPHSAANDPIFVVLHSFTDAIFDEWMKRFNPPADAWPQELAPIGHNRMYNMVPFFPPVTNEELFLTSDQLGYSYAIDLPVSVEETPGWPTTGGGGHFPRACVSSKNLMEKECCPPWSGDRSPCGQLSGRGSCQNILLSNAPLGPQFPFTGVDDRESWPSVFYNRTCQCSGNFMGFNCGNCKFGFWGPNCTERRLLVRRNIFDLSAPEKDKFFAYLTLAKHTISSDYVIPIGTYGQMKNGSTPMFNDINIYDLFVWMHYYVSMDALLGGSEIWRDIDFAHEAPAFLPWHRLFLLRWEQEIQKLTGDENFTIPYWDWRDAEKCDICTDEYMGGQHPTNPNLLSPASFFSSWQIVCSRLEEYNSHQSLCNGTPEGPLRRNPGNHDKSRTPRLPSSADVEFCLSLTQYESGSMDKAANFSFRNTLEGFASPLTGIADASQSSMHNALHIYMNGTMSQVQGSANDPIFLLHHAFVDSIFEQWLRRHRPLQEVYPEANAPIGHNRESYMVPFIPLYRNGDFFISSKDLGYDYSYLQDSDPDSFQDYIKSYLEQASRIWS

SEQ ID NO: 11 - DNA, 4152 bp, Artificial Sequence
  Tri-hybrid antigen coding sequence
atgagtgctcctaaactcctctctctgggctgtatcttcttccccttgctactttttcagcaggcccgggctcaattcccaagacagtgtgccactgttgaggctttgagaagtggtatgtgttgcccagacctgtcccctgtgtctgggcctgggacagaccgctgtggctcatcatcagggaggggcagatgtgaggcagtgactgcagactcccggccccacagccctcagtatccccatgatggcagagatgatcgggaggtctggcccttgcgcttcttcaataggacatgtcactgcaacggcaatttctcaggacacaactgtgggacgtgccgtcctggctggagaggagctgcctgtgaccagagggttctcatagtcaggagaaatcttctggacttaagtaaagaagaaaagaaccactttgtccgggccctggatatggcaaagcgcacaactcaccctttatttgtcattgccaccaggagatcagaagaaatactggggccagatggcaacacgccacaatttgagaacatttccatttataactactttgtttggacacactattactcagtcaaaaagactttccttggggtaggacaggaaagctttggtgaagtggatttctctcatgagggaccagcttttctcacatggcacaggtaccacctcctgcgtctggagaaagacatgcaggaaatgttgcaagagccttctttctcccttccttactggaattttgcaacggggaaaaatgtctgtgatatctgcacggatgacttgatgggatccagaagcaactttgattccactctaataagcccaaactctgtcttttctcaatggcgagtggtctgtgactccttggaagattatgataccctgggaacactttgtaacagcaccgaggatgggccaattaggagaaatccagctggaaatgtggccagaccaatggtgcaacgtcttcctgaaccacaggatgtcgctcagtgcttggaagttggtttatttgacacgcctcctttttattccaactctacaaacagtttccgaaacacagtggaaggttacagtgaccccacgggaaagtatgaccctgctgttcgaagtcttcacaatttggctcatctattcctgaatggaacagggggacaaacccatttgtctccaaatgatcctatttttgtcctcctgcacaccttcacagatgcagtctttgatgaatggctgaggagatacaatgctgatatatccacatttccattggaaaatgcccctattggacataatagacaatacaacatggtgccattctggcccccagtcaccaacacagaaatgtttgttactgctccagacaacctgggatacacttatgaaattcaatggccaagtcgggagtttagtgtacctgagcagttcccccgagtctgcatgacggtggacagcctagtgaacaaggagtgctgcccacgcctgggtgcagagtcggccaatgtctgtggctctcagcaaggccgggggcagtgcacagaggtgcgagccgacacaaggccctggagtggtccctacatcctacgaaaccaggatgaccgtgagctgtggccaagaaaattcttccaccggacctgcaagtgcacaggaaactttgccggctataattgtggagactgcaagtttggctggaccggtcccaactgcgagcggaagaaaccaccagtgattcggcagaacatccattccttgagtcctcaggaaagagagcagttcttgggcgccttagatctcgcgaagaagagagtacaccccgactacgtgatcaccacacaacactggctgggcctgcttgggcccaatggaacccagccgcagtttgccaactgcagtgtttatgatttttttgtgtggctccattattattctgttagagatacattattaggaccaggacgcccctacagggccatagatttctcacatcaaggacctgcatttgttacctggcaccggtaccatttgttgtgtctggaaagagatctccagcgactcattggcaatgagtcttttgctttgccctactggaactttgccactgggaggaacgagtgtgatgtgtgtacagaccagctgtttggggcagcgagaccagacgatccgactctgattagtcggaactcaagattctccagctgggaaactgtctgtgatagcttggatgactacaaccacctggtcaccttgtgcaatggaacctatgaaggtttgctgagaagaaatcaaatgggaagaaacagcatgaaattgccaaccttaaaagacatacgagattgcctgtctctccagaagtttgacaatcctcccttcttccagaactctaccttcagtttcaggaatgctttggaagggtttgataaagcagatgggactctggattctcaagtgatgagccttcataatttggttcattccttcctgaacgggacaaacgctttgccacattcagccgccaatgatcccatttttgtggttcttcattcctttactgatgccatctttgatgagtggatgaaaagatttaatcctcctgcagatgcctggcctcaggagctggcccctattggtcacaatcggatgtacaacatggttcctttcttccctccagtgactaatgaagaactctttttaacctcagaccaacttggctacagctatgccatcgatctgccagtttcagttgaagaaactccaggttggcccacaactcatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtca

SEQ ID NO: 12 - protein, 1384 aa, Artificial Sequence
  Tri-hybrid antigen
MSAPKLLSLGCIFFPLLLFQQARAQFPRQCATVQALRSGMCCPELSPVSGPGTERCGSSSGRGRCQAVTAESRPHSPQYPHEGREERQVWPLRFFNRTCHCNGNFSGHNCGTCRPGWRGAACEQRVLIVRRNLLELSKQQKNHFVRALEMAKRTTHPLFVIATRRSQQILGPEGNTPQFQNISIYNYFVWTHYYSVKKTFLGVGQQSFGQVEFSHQGPAFLTWHRYHLLRLQKEMQQMLQQPSFSLPYWNFATGKNVCEICTEELMGSRSNFESTLISPNSVFSQWRVVCESLQEYETLGTLCNSTQEGPIRRNPAGNVARPMVQRLPQPQEVAQCLQVGLFETPPFYSNSTNSFRNTVQGYSEPTGKYEPAVRSLHNLAHLFLNGTGGQTHLSPNEPIFVLLHTFTEAVFEQWLRRYNAEISTFPLQNAPIGHNRQYNMVPFWPPVTNTQMFVTAPENLGYTYQIQWPSRQFSVPQQFPRVCMTVESLVNKQCCPRLGAQSANVCGSQQGRGQCTQVRAETRPWSGPYILRNQEERQLWPRKFFHRTCKCTGNFAGYNCGECKFGWTGPNCQRKKPPVIRQNIHSLSPQQRQQFLGALELAKKRVHPEYVITTQHWLGLLGPNGTQPQFANCSVYEFFVWLHYYSVRETLLGPGRPYRAIEFSHQGPAFVTWHRYHLLCLQRELQRLIGNQSFALPYWNFATGRNQCEVCTEQLFGAARPEEPTLISRNSRFSSWQTVCESLEEYNHLVTLCNGTYQGLLRRNQMGRNSMKLPTLKEIRECLSLQKFENPPFFQNSTFSFRNALQGFEKAEGTLESQVMSLHNLVHSFLNGTNALPHSAANEPIFVVLHSFTEAIFEQWMKRFNPPAEAWPQQLAPIGHNRMYNMVPFFPPVTNQQLFLTSEQLGYSYAIELPVSVQQTPGWPTTHFPRACVSSKNLMQKQCCPPWSGERSPCGQLSGRGSCQNILLSNAPLGPQFPFTGVEERQSWPSVFYNRTCQCSGNFMGFNCGNCKFGFWGPNCTQRRLLVRRNIFELSAPQKEKFFAYLTLAKHTISSEYVIPIGTYGQMKNGSTPMFNEINIYELFVWMHYYVSMEALLGGSQIWREIEFAHQAPAFLPWHRLFLLRWQQQIQKLTGEQNFTIPYWEWREAQKCEICTEQYMGGQHPTNPNLLSPASFFSSWQIVCSRLQQYNSHQSLCNGTPQGPLRRNPGNHEKSRTPRLPSSAEVQFCLSLTQYQSGSMEKAANFSFRNTLQGFASPLTGIAEASQSSMHNALHIYMNGTMSQVQGSANEPIFLLHHAFVESIFQQWLRRHRPLQQVYPQANAPIGHNRQSYMVPFIPLYRNGEFFISSKELGYEYSYLQESEPESFQEYIKSYLQQASRIWS

SEQ ID NO: 13 - protein, 529 aa, human [sequence omitted]

SEQ ID NO: 14 - protein, 537 aa, human
  TRP-1 (GenBank Accession No. NP_000541)
MSAPKLLSLGCIFFPLLLFQQARAQFPRQCATVEALRSGMCCPDLSPVSGPGTDRCGSSSGRGRCEAVTADSRPHSPQYPHDGRDDREVWPLRFFNRTCHCNGNFSGHNCGTCRPGWRGAACDQRVLIVRRNLLDLSKEEKNHFVRALDMAKRTTHPLFVIATRRSEEILGPDGNTPQFENISIYNYFVWTHYYSVKKTFLGVGQESFGEVDFSHEGPAFLTWHRYHLLRLEKDMQEMLQEPSFSLPYWNFATGKNVCDICTDDLMGSRSNFDSTLISPNSVFSQWRVVCDSLEDYDTLGTLCNSTEDGPIRRNPAGNVARPMVQRLPEPQDVAQCLEVGLFDTPPFYSNSTNSFRNTVEGYSDPTGKYDPAVRSLHNLAHLFLNGTGGQTHLSPNDPIFVLLHTFTDAVFDEWLRRYNADISTFPLENAPIGHNRQYNMVPFWPPVTNTEMFVTAPDNLGYTYEIQWPSREFSVPEIIAIAVVGALLLVALIFGTASYLIRARRSMDEANQPLLTDQYQCYAEEYEKLQNPNQSVV

SEQ ID NO: 15 - protein, 519 aa, human
  TRP-2(GenBank Accession No. NP_001913)
MSPLWWGFLLSCLGCKILPGAQGQFPRVCMTVDSLVNKECCPRLGAESANVCGSQQGRGQCTEVRADTRPWSGPYILRNQDDRELWPRKFFHRTCKCTGNFAGYNCGDCKFGWTGPNCERKKPPVIRQNIHSLSPQEREQFLGALDLAKKRVHPDYVITTQHWLGLLGPNGTQPQFANCSVYDFFVWLHYYSVRDTLLGPGRPYRAIDFSHQGPAFVTWHRYHLLCLERDLQRLIGNESFALPYWNFATGRNECDVCTDQLFGAARPDDPTLISRNSRFSSWETVCDSLDDYNHLVTLCNGTYEGLLRRNQMGRNSMKLPTLKDIRDCLSLQKFDNPPFFQNSTFSFRNALEGFDKADGTLDSQVMSLHNLVHSFLNGTNALPHSAANDPIFVVLHSFTDAIFDEWMKRFNPPADAWPQELAPIGHNRMYNMVPFFPPVTNEELFLTSDQLGYSYAIDLPVSVEETPGWPTTLLVVMGTLVALVGLFVLLAFLQYRRLRKGYTPLMETHLSSKRYTEEA

SEQ ID NO: 16 - DNA, 42 bp, Artificial Sequence
  Amplification primer
gcggcggaattccaattcccaagacagtgtgccactgttgag

(19) United States
(12) Patent Application Publication
(10) Pub. No.: US 2004/0132972 A1
(43) Pub. Date: Jul. 8, 2004

(54) Tri-hybrid melanoma antigen

(76) Inventors: Xiaoqiang Kang, Plainsboro, NJ (US); Daniel J. Hicklin, Upper Montclair, NJ (US)

Correspondence Address: KENYON & KENYON, ONE BROADWAY, NEW YORK, NY, 10004, US

(21) Appl. No.: 10/469,217

(22) PCT Filed:
(86) PCT No.: PCT/US02/06034

(51) Int. Cl. A61K 38/17; C07H 21/04; C07K 14/47

(52) U.S. Cl. 530/350; 514/12; 435/69.1; 435/320.1; 435/325; 536/23.2

(57) ABSTRACT

The present invention relates to novel tri-hybrid melanoma antigens, including antigenic fragments or derivatives thereof, of a tyrosinase (TYR) antigen, a tyrosinase-related protein 1 (TRP-1) antigen, and a tyrosinase-related protein 2 (TRP-2) antigen and nucleic acids encoding them. The novel tri-hybrid melanoma antigens of the present invention are useful in the diagnosis, treatment and prevention of human neoplasms, including malignant tumors, such as carcinomas, sarcomas, leukemia, and lymphomas, and pre-malignant lesions, such as adenomas and dysplastic lesions.

### FIELD OF THE INVENTION

The present invention relates to novel tri-hybrid melanoma antigens, including antigenic fragments or derivatives thereof, of a tyrosinase (TYR) antigen, a tyrosinase related protein-1 (TRP-1) antigen, and a tyrosinase related protein-2 (TRP-2) antigen and nucleic acids encoding them, which are useful in the prevention and treatment of human neoplasms.

### BACKGROUND OF THE INVENTION

Cancer cells are not static in nature, but are changing constantly. To escape immunosurveillance and multiply, cancer cells have developed a number of different mechanisms, include the following: decreasing expression in MHC molecules (see Ferrone et al., Immunol. Today, 16:487-494 (1995)); deficiencies in antigen processing (see Kamarashev et al., Int. J. Cancer, 95:23-28 (2001)); secretion of immune suppressing cytokines (see Spellman et al., Surg Oncol., 5(5-6):237-44 (1996)); and loss of tumor antigen expression (see Ohmacht et al., J. Cell Physiol., 182: 332-338 (2000)).

With respect to the loss of tumor antigen expression, a number of melanoma-specific antigens have been identified in recent years. One major group of such antigens, which are recognized by the immune system, consists of melanocyte differentiation antigens, such as tyrosinase, TRP-1 (also designated gp75), TRP-2, gp100 and MART-1/Melan-A. All of these antigens are present in melanosomes.

Tyrosinase, TRP-1, and TRP-2, are melanocyte differentiation antigens, located in the melanosomes of melanocytes and melanomas, and involved in melanin synthesis. See Kawakami et al., Immunol. Res., 16: 313-339 (1997); Kawakami et al., J. Immunother., 21: 237-246 (1998). Human tyrosinase, a 529 amino acid melanosomal membrane protein, has tyrosine hydroxylase, DOPA oxidase and DHI activity, and is the principal enzyme involved in melanin synthesis. Human TRP-1 consists of 537 amino acids and has DHI-2-carboxylic acid oxidase activity. Human TRP-2 is a 519 amino acid melanosomal enzyme with DOPAchrome tautomerase activity. Antibodies and T cells to these antigens have been identified in melanoma patients. See Houghton et al., Ann. N.Y. Acad. Sci., 690: 59-68 (1993); Brichard et al., J. Exp. Med., 178: 489-495 (1993); Kang et al., J. Immunol., 155: 1443-1348 (1995); Wang et al., J. Exp. Med., 181: 799-804 (1995); Wang et al., J. Exp. Med., 184: 2207-2216 (1996). However, it remains unclear how tolerance to these differentiation antigens is broken in cancerous state.

Animal models of cancer vaccines have demonstrated the feasibility of melanoma vaccines using the tyrosinase family of proteins. Vaccination of mice with recombinant vaccinia viruses expressing mTRP-1 (see Overwijk et al., Proc. Nat'l Acad. Sci. USA., 96: 2982-2987 (1999)), or naked DNA encoding hTRP-1 (see Weber et al., J. Clin. Invest., 102: 1258-1264 (1998)), or insect cells containing mTRP-1 (see Naftger et al., Proc. Nat'l Acad. Sci. USA., 93: 14809-14814 (1996)) have demonstrated the ability to break tolerance to TRP-1 and have shown induction of strong antitumor activities, which were dependent on TRP-1 specific antibodies and CD4 T cells. Immune tolerance to TRP-2 also has also been broken with naked DNA encoding human TRP-2, following induction of CD8 T cells (see Browne et al., J. Exp. Med., 190: 1717-1722 (1999)).

Many of the clinical vaccine trials targeting the tyrosinase family of proteins are underway to treat melanoma. It has been reported that melanoma cells can lose the expression of TRP-1, TRP-2, or tyrosinase. See Id., Marincola et al., Adv. Immunol., 74: 181-273 (2000). However, the likelihood that a melanoma will lose all three antigens at same times is low; therefore, a chimeric protein containing all three of these melanoma antigens will have great advantage.

Most of the vaccine approaches are based on defined peptide epitopes, plasmid DNA, and recombinant viruses, each of which suffers from various disadvantages. The identification of HLA-binding epitopes and HLA types of each patient are needed for peptide epitope-based vaccine. Plasmid DNAs in general are week immunogens. Recombinant viruses generate neutralizing antibodies, which limit the administration of vaccine. As such, use of a recombinant protein vaccine is an attractive alternative approach for a cancer vaccine.

There are several advantages to use of a recombinant protein vaccine, including the fact that the vaccine can be administered repeatedly, it can induce a wise spectrum of immune responses, including production of antibodies, cytotoxic T-lymphocytes (CTLs) (with appropriate adjuvants), and CD4+ T cells (important in maintaining tumor immunity). Moreover, a protein vaccine can contain all possible epitopes of the antigen.

### SUMMARY OF THE INVENTION

The present invention is directed to tri-hybrid melanoma antigens, and isolated DNAs encoding such antigens, comprising tyrosinase or a fragment thereof, TRP-1 or a fragment thereof, or TRP-2 or a fragment thereof. The present invention is also directed to compositions for inhibiting melanosomal activity or tumor growth comprising such tri-hybrid melanoma antigens and isolated DNAs. In addition, the present invention is directed to methods of eliciting an immune response against a melanosomal antigen, methods of treating a tumor, or methods of vaccination using such tri-hybrid melanoma antigens and isolated DNAs.

### DESCRIPTION OF THE FIGURES

FIG. 1 shows construction of a tri-hybrid melanoma antigen, hTRPx3, containing human TRP-1, TRP-2, and tyrosinase. A tri-hybrid melanoma antigen DNA fragment was generated with primers and the resulting fragments were mixed and fused following 10 PCR cycles. The final chimeric DNA was generated with primers htyrF-1 (SEQ ID NO:1) and htyrF-6 (SEQ ID NO:6) and then cloned into bacterial expression vector pET28a(+).

FIG. 2 shows purification and characterization of a recombinant tri-hybrid melanoma antigen, hTRPx3 protein, by SDS-PAGE and Western blotting. In FIG. 2A, an SDS-PAGE gel shows the expression and purification of the hTRPx3 protein and in FIG. 2B, a Western blot shows that the purified hTRPx3 protein is recognized by antibodies specific for human TRP-1, TRP-2, and tyrosinase.

FIG. 3 graphically demonstrates the antibody responses in mice following immunization with a tri-hybrid melanoma antigen, hTRPx3 protein. In FIG. 3A, a graph shows that hTRPx3 protein immunization induced antibodies against TRP-1, TRP-2, and tyrosinase and hTRPx3. In FIG. 3B, a graph shows the isotypes of the antibodies specific to the hTRPx3 protein.

FIG. 4 graphically demonstrates that immunization of mice with a tri-hybrid melanoma antigen, hTRPx3, resulted in production of IFNγ releasing T cells specific for TRP-1, TRP-2, and tyrosinase.

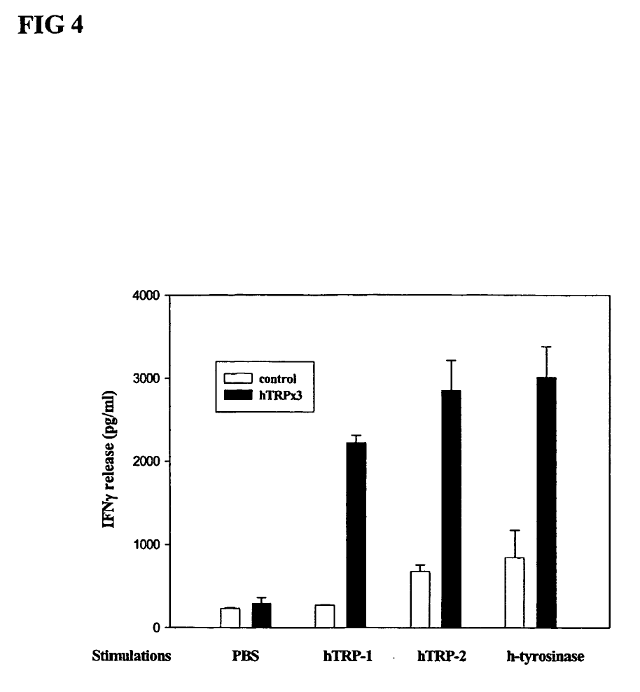

FIG. 5 shows induction of a T cell immune response following immunization with a tri-hybrid melanoma antigen, hTRPx3 protein. In FIG. 5A, an ELISPOT blot shows induction of IFNγ-producing T cells, and in FIG. 5B, this IFNγ-producing T cell induction is represented graphically.

FIG. 6 shows that immunization with a tri-hybrid melanoma antigen, hTPRx3 protein, was useful in treating tumors in mammals. In FIG. 6A, the number of lung surface metastases following immunization is represented graphically. In FIG. 6B, the number of lung surface metastases following immunization of MHC class I knock-out mice, MHC class II knock-out mice, and FcRγ knock-out mice is represented graphically.

### DETAILED DESCRIPTION OF THE INVENTION

The present invention relates to tri-hybrid melanoma antigens of a tyrosinase antigen (U.S. Pat. No. 4,898,814), a TRP-1 antigen (WO 91/14775) and a TRP-2 antigen (U.S. Pat. No. 5,831,016), including antigenic fragments and derivatives thereof. Derivatives include, for example, antigens that are mutational or allelic variants. The antigens can be of human or, more generally, of mammalian origin, and the components of the tri-hybrid melanoma antigen can be from different sources (e.g., homologous antigens from different species). Moreover, each component (i.e., antigen or antigenic fragment) can be a hybrid combining sequences from more than one source. The invention provides a tri-hybrid melanoma antigen that is more effective for immunization against melanoma than any single antigen from which it is derived.

“Antigenic fragment,” as the term is used herein, means any antigenic segment of a protein or gene, usually having at least 5 or 6 amino acids in the case of a protein fragment and at least 15-18 nucleotides in the case of a gene. Thus, a fragment generally encompasses a segment of a protein, or the nucleotide sequence that encodes it, which comprises at least one B cell or T cell epitope.

Tyrosinase, TRP-1 (also known as gp75) and TRP-2 are expressed primarily in melanomas, normal melanocytes, and in the retina. The three proteins are related in sequence, sharing (pairwise) greater than 40% amino acid sequence identity and greater than 50% amino acid sequence similarity, and have in common N-terminal signal peptides and a C-terminal sequence involved in intracellular retention and sorting to melanosomes along the endosomal/lysosomal pathway. Moreover, these three members of the tyrosinase family of proteins are highly conserved among species.

The invention further relates to homologs of human tyrosinase, TRP-1 and TRP-2, that can be used to break tolerance in humans to the human proteins. Table 1 provides two examples of such homologs for each of these three tyrosinase family proteins. The value for percent identity of the homolog to the human protein is calculated over the entire length of the protein. Homologs that can be used according to the invention have at least 60% identity to the corresponding protein of the species in which tolerance is to be broken. Preferred homologs have at least 70% identity. More preferred homologs have at least 80% identity. It is noted that where less than a complete amino acid sequence is to be incorporated into a tri-hybrid melanoma antigen, percent identity is calculated only over the length of the protein fragment that is incorporated.

Nucleotide sequences of mRNAs encoding human tyrosinase, TRP-1/gp75 and TRP-2 and the sequences of the proteins themselves are publicly available from GenBank (National Center for Biotechnology Information, National Library of Medicine, Bethesda, Md.), as are homologous sequences from other species. Nucleotide and amino acid sequences referred to herein correspond to GenBank accession numbers as given in Table 1. The sequences give therein are meant as examples only, and do not limit the scope of the invention.

TABLE 1

| | | Nucleotide | Amino Acid | Percent Identity |
|---|---|---|---|---|
| | Source | Sequence | Sequence | to Human Protein |
| Tyrosinase | Human | NM_000372 | NP_000363 | — |
| | Mouse | NM_011661 | NP_035791 | 86 |
| | Rabbit | AF210660 | AAF43895 | 89 |
| TRP-1 | Human | NM_000550 | NP_000541 | — |
| | Mouse | MMTYRR | TYR1_MOUSE | 85 |
| | Goat | AF136926 | AAD34802 | 88 |
| TRP-2 | Human | NM_001922 | NP_001913 | — |
| | Mouse | NM_010024 | NP_034154 | 83 |
| | Chicken | AF023471 | AAC63434 | 69 |

A preferred tri-hybrid melanoma antigen of the present invention comprises the soluble portion of each of tyrosinase, TRP-1 and TRP-2. For example, SEQ ID NO:7, SEQ ID NO:9, and SEQ ID NO:11 provide examples of DNA sequences encoding a tri-hybrid melanoma antigen in the context of the present invention. The corresponding protein sequence for the tri-hybrid melanoma antigen is set forth in SEQ ID NO:8, SEQ ID NO:10, and SEQ ID NO:12. Accordingly, in one embodiment, an isolated DNA encoding the tri-hybrid melanoma antigen comprises SEQ ID NO:7 or a fragment thereof, SEQ ID NO:9 or a fragment thereof, or SEQ ID NO:11 or a fragment thereof and the tri-hybrid melanoma antigen itself comprises SEQ ID NO:8 or a fragment thereof, SEQ ID NO:10 or a fragment thereof, or SEQ ID NO:12 or a fragment thereof.

An example of a more preferred tri-hybrid melanoma antigen comprises the sequence from about amino acid residue 25 to about amino acid residue 477 of human TRP-1 or a homolog thereof, the sequence from about amino acid residue 24 to about amino acid residue 472 of human TRP-2 or a homolog thereof, and the sequence from about amino acid residue 19 to about amino acid residue 476 of human tyrosinase or a homolog thereof. The individual fragments can be linked in any order and the tri-hybrid melanoma antigen can further comprise glycine residues by which the fragments are linked.

Thus, a particularly preferred tri-hybrid melanoma antigen is represented by SEQ ID NO:8 from about amino acid residue number 25 to about amino acid residue number 1392 (containing glycine linkages) and SEQ ID NO:12 from about amino acid residue number 25 to about amino acid residue number 1384 (without glycine linkages). It will be apparent to one of ordinary skill in the art that there can be some variation in the extent of the protein fragments of which the tri-hybrid melanoma antigen is comprised, which will have little or no effect on its immunogenic properties.

Where non-human mammalian proteins, mutational variants, hybrids, fragments, or derivatives are used, they can be selected such that they possess desirable properties such as increased immunogenicity, decreased side effects, and increased half-life. For example, fragments of the individual antigens can be selected for incorporation into a tri-hybrid melanoma antigen of the invention based on the presence of known or postulated B cell or T cell epitopes. As another example, mutational variants that exhibit improved binding to MHC molecules can be selected.

Production of large quantities of tri-hybrid melanoma antigens can be accomplished by methods known in the art. For example, large amounts of tri-hybrid melanoma antigens can readily be synthesized in vitro. As another example, nucleic acid encoding tri-hybrid melanoma antigens can be transfected into bacterial, insect, or mammalian cells using appropriate vectors and methods as known in the art. Accordingly, the present invention encompasses cloning or expression vectors comprising a DNA encoding a tri-hybrid melanoma antigen and host cells comprising such cloning or expression vectors.

Where needed or desired for expression of the tri-hybrid melanoma antigen in a given cell type, the first protein fragment of the tri-hybrid melanoma antigen will be preceded by a signal peptide, which can be the signal peptide that is native to the first protein fragment in the tri-hybrid melanoma antigen, or can be a signal peptide derived from another source. For example, a signal sequence signaling for secretion is useful where it is desired to provide for secretion of a tri-hybrid melanoma antigen into external mileau of a cell. For a review of secretion and signal peptide function, see, for example, Pugsley, Curr. Opin. Cell Biol., 2:609-616 (1990). Algorithms and computer implementations for secretory signal sequence prediction are available. See, e.g., von Heijne et al., Nucleic Acids Res., 14:4683-4690 (1986); Nielsen et al., Protein Eng., 10:1-6 (1997).

For expression in bacterial cells, a bacterial signal sequence can be preferred. Alternatively, a signal sequence is not necessary to express the tri-hybrid melanoma antigen in a bacterial host in inclusion bodies. For example, a tri-hybrid melanoma antigen of the invention can comprise the amino acid sequence represented by SEQ ID NO:8 from about amino acid residue number 25 to about amino acid residue number 1392. Such a tri-hybrid melanoma antigen can be expressed with an N-terminal methionine residue, depending on the nature of the host bacteria. The methionine can be cleaved in vivo in the bacterial host cell or remain intact. Such a tri-hybrid melanoma antigen can be expressed in E. coli and obtained from inclusion bodies by methods well known in the art.

To assist in purification, tri-hybrid proteins of the invention can be expressed with fused “tag” sequences. For example, the tri-hybrid melanoma antigen encoding DNA sequence can be cloned in an expression vector that provides for production of the tri-hybrid melanoma antigen linked to an N-terminal His tag sequence. The His tag allows purification by metal chelation chromatography. In certain embodiments, the His tag can be cleaved from the tri-hybrid melanoma antigen after purification. Alternatively, tag sequences that provide for affinity purification can be used. In a preferred embodiment, a tri-hybrid melanoma antigen is expressed from pET-28a(+), purified by metal chelation chromatography, and the His tag removed by specific proteolysis at the thrombin cleavage site.

The invention provides novel tri-hybrid melanoma antigens that are particularly useful as vaccines for inducing immune responses effective for treating, inhibiting, and preventing cancers and precancers. Of particular interest are tri-hybrid melanoma antigens that induce anti-tumor immune responses in patients with melanoma. Accordingly, the present tri-hybrid melanoma antigens can be administered for prophylactic and/or therapeutic treatments of various conditions. Treatment, in the context of the present invention, is intended to encompass inhibiting, slowing, or reversing the progress of the underlying condition, ameliorating clinical symptoms of a condition or preventing the appearance of clinical symptoms of the condition.

The term “melanoma” includes, but is not limited to, melanomas, metastatic melanomas, melanomas derived from either melanocytes or melanocyte related nevus cells, melanocarcinomas, melanoepitheliomas, melanosarcomas, melanoma in situ, superficial spreading melanoma, nodular melanoma, lentigo malignant melanoma, acral lentiginous melanoma, invasive melanoma and familial atypical mole and melanoma (FAM-M) syndrome.

Many methods suitable for administering the tri-hybrid melanoma antigen are known in the art. For example, the tri-hybrid melanoma antigen can be administered in soluble form. As another example, autologous mammalian cells capable of expressing the tri-hybrid melanoma antigen can be administered. As another example, virus having tri-hybrid melanoma antigen on its surface can be administered. As yet another example, virus carrying nucleic acid encoding the tri-hybrid melanoma antigen can be administered. As still another example, naked DNA or other nucleic acid encoding the tri-hybrid melanoma antigen can be administered.

Tri-hybrid melanoma antigens can be administered alone, combined with adjuvants, linked to helper (carrier) peptides, proteins, lipids or liposomes, or pulsed onto purified antigen presenting cells (APCs) and the antigen presenting cells used for immunization. Adjuvants for use in immunization and other treatment methods include, for example, RIBI Detox (Ribi Immunochemical), QS2 1, CRIS-2 1, alum, BCG and incomplete Freund's adjuvant. For test animals, adjuvants further include complete Freund's adjuvant and others commonly used in the art. Tri-hybrid melanoma antigens can be also be complexed with heat shock binding proteins. APCs are generally eukaryotic cells with major histocompatibility complex (MHC), either class I or class II, gene products at their cell surface. Some examples of APCs that can be used in the present invention include DC, as well as macrophages, preferably MHC class II positive macrophages, monocytes, preferably MHC class II positive monocytes, and lymphocytes. See generally U.S. Pat. No. 5,597,563. It should be appreciated that such administration can be carried out before, simultaneously with, or after administration of the novel tri-hybrid melanoma antigens.

Tri-hybrid melanoma antigens can be administered as DNA vaccines. DNA vaccines can comprise “naked” DNA encoding tri-hybrid melanoma antigens. Preferably, the tri-hybrid melanoma antigen-encoding DNA is taken up by host cells and expressed polypeptides are efficiently presented to the immune system. For example, naked DNA can be injected intradermally or intramuscularly or linked to lipids. Preferably, vaccines comprising tri-hybrid melanoma antigen injected directly into muscle or into the skin raise both cellular and humoral immune reactions to encoded antigens. See, for example, U.S. Pat. No. 5,831,016, Gregersen, Naturwissenschaften, 88(12):504-13 (December 2001), and Wang et al., Expert Opin. Biol. Ther., 1(2):277-90 (March 2001) for methods of preparation and use of DNA vaccines. Vaccines can comprise non-viable DNA vectors comprising DNA encoding tri-hybrid melanoma antigen of the present invention. Non-viable DNA vectors have the advantage of ease of preparation and safety of administration. DNA sequences encoding tri-hybrid melanoma antigens of the present invention can be administered using a gene gun in amounts to elicit a cellular response against a cancer cell. Nanogram quantities can be useful for such purposes.

DNA encoding tri-hybrid melanoma antigens can also be expressed by bacteria or from recombinant viruses upon infection of host cells of the patient. Such bacterial or viral vectors can be designed to also express co-immunostimulatory molecules which enhance an immune response. Co-immunostimulatory molecules include, for example, IL-2, IL-6, IL-10, IL-12, and γ-interferon (IFN-γ). Co-immunostimulatory molecules can be selected so as to favor humoral immune responses or cytotoxic immune responses.

DNA encoding tri-hybrid melanoma antigens of the present invention can also be used to create genetically modified immune cells capable of recognizing human tumor antigens. Such genetically modified immune cells can be particularly effective in, for example, mediating the regression of cancer in selected patients with metastatic melanoma. Techniques by which human lymphocytes are sensitized in vitro to tumor antigen peptides presented on antigen presenting cells are known in the art. By repetitive in vitro stimulation cells can be derived with a far greater capacity to recognize and respond to human tumor antigens. Thus by repeated in vitro sensitization with the tumor antigen of the present invention, lymphocytes can be derived with increased potency. The cells to be sensitized can be obtained from the subject to be treated or can be MHC matched cells from other sources. Adoptive transfer of these cells into the subject to be treated can result in increased effectiveness in mediating tumor regression in vivo.

Tri-hybrid tumor antigens can be administered via one or more of several routes including, but not limited to, intravenous, intramuscular, subcutaneous, intradermal, intraperitoneal, intrathecal, intrapleural, intrauterine, rectal, vaginal, topical, intratumor and the like.

Administration can be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents can be used to facilitate permeation. Transmucosal administration can be by nasal sprays, for example, or suppositories. For oral administration, the tumor antigen, cancer peptides or variants thereof are formulated into conventional oral administration forms such as capsules, tablets and tonics.

It is understood that the tri-hybrid melanoma antigens of the present invention, where used in an animal for the purpose of prophylaxis or treatment, can be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers can further comprise minor amounts of auxiliary substances, such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The compositions of this invention can be in a variety of forms. These include, for example, solid, semi-solid and liquid dosage forms, such as tablets, pills, powders, liquid solutions, dispersions or suspensions, liposomes, suppositories, injectable and infusible solutions. The preferred form depends on the intended mode of administration and therapeutic application.

Such compositions of the present invention are prepared in a manner well known in the pharmaceutical art. In making the composition the active ingredient will usually be mixed with a carrier, or diluted by a carrier and/or enclosed within a carrier which can, for example, be in the form of a capsule, sachet, paper or other container. When the carrier serves as a diluent, it can be a solid, semi-solid, or liquid material, which acts as a vehicle, excipient or medium for the active ingredient. Thus, the composition can be in the form of tablets, lozenges, sachets, cachets, elixirs, suspensions, aerosols (as a solid or in a liquid medium), ointments containing, for example, up to 10% by weight of the active compound, soft and hard gelatin capsules, suppositories, injection solutions, suspensions, sterile packaged powders and as a topical patch.

It should be appreciated that the immunogen of the present invention can be administered to any suitable animal. For example, the animal is preferably a mammal, such as a rabbit, rat, or mouse. More preferably, the animal is a human.

The tri-hybrid tumor antigen according to the present invention is preferably provided in a therapeutically effective amount. Preferably, the dose is effective to prime, stimulate and/or cause the clonal expansion of cancer antigen specific B and T lymphocytes, which in turn are capable of preventing, inhibiting, or treating cancer in the recipient.

Therapeutically effective doses can be determined by those skilled in the relevant arts via clinical studies. Therapeutically effective doses can also be determined by in appropriate animal models, then extrapolated to humans using known techniques. For example, for systemic administration, the amount of compound administered per unit body weight determined in rats can easily be applied to humans.

Therapeutically effective doses will vary, depending on such factors as the weight and condition of the patient, the type of melanoma or other cancer to be treated, inhibited, or prevented, and the method of administration.

The dosage of tri-hybrid tumor antigen for a human can be at least about 1 pg per Kg of body weight. A range of from about 1 ng per Kg body weight to about 100 mg per Kg body weight is preferred. More preferably, the amount administered can be at least about 1 □g per Kg body weight to about 1 mg body weight.

The dose is administered at least once and can be provided as a bolus or a continuous administration. Multiple administrations of the dose over a period of several weeks to months can be preferable. Where multiple doses are provided, the dosage amount and formulation can be the same or can differ among doses.

Effective treatment of melanoma or other cancer or pre-malignant lesion can be measured by many parameters known in the art, including, but not limited to, the decrease of tumor burden, increase in humoral immune response, changes in serum tumor markers, and increase in cytotoxic or cell-mediated immune responses. As more specific examples, antibody levels and CTL levels can be measured. Still other examples of criteria that can be used to evaluate effective treatment include standard World Health Organization (WHO) criteria for tumor response. Effective prevention of melanoma can be measured by many parameters known in the art, including, but not limited to, the decrease of incidence in a treated population compared to an untreated population. Such criteria can be measured by methods known in the art.

Accordingly, the present invention can be used in vivo and in vitro for investigative, diagnostic, prophylactic, or treatment methods, which are well known in the art.

Of course, it is to be understood and expected that variations in the principles of the invention herein disclosed can be made by one skilled in the art and it is intended that such modifications are to be included within the scope of the present invention.

All references mentioned herein are incorporated in their entirety.

### EXAMPLES

The examples that follow further illustrate the invention, but should not be construed to limit the scope in any way. Detailed descriptions of conventional methods, such as those employed in the construction of vectors and plasmids, the insertion of genes encoding polypeptides into such vectors and plasmids, the introduction of plasmids into host cells, and the expression and determination thereof of genes and gene products can be obtained from numerous publications, including Sambrook, J. et al., (1989) Molecular Cloning: A Laboratory Manual, 2nd ed., Cold Spring Harbor Laboratory Press.

General Methods

Cells and Animals

The mouse melanoma cell line B16BL6 was kindly provided by Dr. Isaiah Fidler (M. D. Anderson Cancer Center, Houston); B16BL6 expression of TRP-1, TRP-2, and tyrosinase was determined by RT-PCR and Western analysis. The EL4 cell line was obtained from American Type Culture Collection (Manassas, Va.). C57BL/6 and C57BL/6 with deficiencies in MHC class I, or MHC class II, or FcR were purchased from Jackson Laboratory (Bar Harbor, Me.). All cell lines were maintained in RPMI 1640 (Gibco BRL, Gaithersburg, Md.) with 10% heat-inactivated fetal bovine serum and without antibiotics and were routinely tested for Mycoplasma contamination and were negative.

All experiments and procedures were performed in accordance with the United States Department of Agriculture and Human Services, and NIH policies regarding the use of laboratory animals.

Generation of a DNA Fragment Containing hTRP-1, hTRP-2, and h-Tyrosinase

To generate a chimeric DNA fragment, three pairs of primers were designed. Primer pair one used hgp75 as template, primer pair two used hTRP-2 as template, and primer pair three used h-tyrosinase as template. Pair one, termed htyrF-1 and htyrF-2, generated a DNA fragment containing a soluble hgp75, four-glycines linker and the 5′ portion of hTRP-2 (htyrF-1 (SEQ ID NO:1): 5′ gccgaatcca tgagtgctcc taaactcctc 3′ and htyrF-2 (SEQ ID NO:2): 5′ cgtcatgcag actcggggga actgccctcc gccaccctca ggtacactaa actcccgact tgg 3′). Pair two, termed htyr-3 and htyr-4, cover the 3′ portion of hgp75, the soluble portion of hTRP-2, four glycines linker, and the 5′ portion of h-tyrosinase (htyrF-3 (SEQ ID NO:3): 5′ ccaagtcggg agtttagtgt acctgagggt ggcggagggc agttcccccg agtctgcatg acg 3′ and htyrF-4 (SEQ ID NO:4): 5′ ggagacacag gctctaggga aatgtccacc cccgccagtt gtgggccaac ctggagtttc 3′). Pair three, termed htyr-5 and htyr-6, PCR-cloned a fragment containing the 3′ portion of h-tyrosinase, the soluble portion of h-tyrosinase, and the stop codon (htyrF-5 (SEQ ID NO:5): 5′ gaaactccag gttggcccac aactggcggg ggtggacatt tccctagagc ctgtgtctcc 3′ and htyr-6 (SEQ ID NO:6): 5′ gcggcgctcg agctatgacc agatccgact cgcttg 3′). Each PCR reaction resulted in a 1.4 kb fragment (PCR product one). The resulting three PCR products were then mixed and underwent 10 cycle PCR reactions (PCR product two). Finally, chimeric DNA was generated with primers htyr-1 and htyr-6 (PCR product three). PCR product three was about 4.1 kb and digested with EcoR I and Xho I before cloning into pET28a(+) (Novogen, Madison, Wis.).

Expression and Purification of Recombinant hTRPx3

E. coli BL21 (Novogen, Madison, Wis.) transformed with pET28a(+) containing hTRPx3 cDNA were grown to a cell density of 0.6 (A₆₀₀ nm) at 37° C. in shake flasks. The lac promoter was induced by addition of IPTG to a final concentration of 1 mM, and cells were grown for an additional 4 hrs at 37° C. The cells were harvested by centrifugation, resuspended in PBS and disrupted with a Cell Disrupter (Constant Systems Ltd, Warwick, UK). The insoluble fraction of the E. coli homogenate, which contained recombinant inclusion bodies, was harvested by centrifugation (10,000 g, 4° C., 30 min). The pellets were dissolved in a solution containing 8M urea, 50 mM Tris, and 5 mM imidazole at pH 8.0. The recombinant hTRPx3 were affinity purified using Toyopearl His binding resin according to manufacturer's instruction (TosoHaas, Montgomeryville, Pa.). The recombinant human TRP-1, TRP-2, tyrosinase was expressed and purified in a similar manner.

Western blots were performed to ascertain that the recombinant hTRPx3 was recognized by antibodies specific for hTRP-1, hTRP-2, and h-tyrosinase. The purified hTRPx3 protein was run on 12% SDS-PAGE and transferred to PVDF membranes (Novex, San Diego, Calif.). The membranes were blocked overnight at 4° C. in 5% non-fat dry milk (blocking buffer) and probed with mouse sera specifically for individual antigens diluted 1:500 in PBS-0.2% Tween-20 at room temperature for 1 hr with gentle agitation. Blots were then washed with PBS-0.2%Tween-20 solution and incubated with peroxidase-conjugated goat anti-mouse IgG (Biosource, Camarillo, Calif.) diluted in PBS-0.2% Tween-20 (1:5000) for 1 hr. Blots were washed extensively as above and detected via ELC Western blotting reagents (Amersham Pharmacia Biotech, Little Chalfont, UK). Mouse sera specific for hTRP-1, hTRP-2, and h-tyrosinase were generated from mice immunized with individual proteins.

Enzyme-Linked Immunosorbent Assay (ELISA)

An indirect ELISA was developed to detect antibody responses. Purified human protein antigens, 50 ng in 50 μl PBS buffer, was added to each well of a 96-well Immunolon-2 plate (Dynex Technologies, Chantilly, Va.) and incubated overnight at 4° C. Plates were washed twice with PBS-0.2% Tween-20 and then incubated in blocking buffer (PBS containing 5% non-fat dry milk) at room temperature for 2 hrs. Mouse sera diluted at different concentrations in PBS-0.2% Tween-20 were added to plates and incubated at room temperature for 2 hrs. Wells were washed three times as above and then 100 μl peroxidase conjugated goat anti-mouse IgG (Biosource, Camarillo, Calif.) diluted 1:5000 in PBS-0.2% Tween-20 was added and incubated for 1 hr at room temperature. Wells then were washed three times and the peroxidase substrate TMB (KLP, Gaithersburg, Md.) was added. The absorbance at 450 nm was read on a kinetic microplate plate reader (Molecular Devices, Sunnyvale, Calif.).

IFNγ Release Assay

Splenocytes (4×10⁵) were harvested one week after the last immunization and cultured with 10 mg/ml recombinant proteins in a total volume of 2 ml of RPMI 1640 with 10% fetal bovine serum in a 24 well plate for 72 hrs. The supernatant were harvested and tested for IFNγ using ELISA kits (Research Diagnostics Inc., Flanders, N.J.).

ELISPOT Assay

To conduct ELISPOT assay, 1×10⁴ fresh isolated spleen cells from each vaccinated mice group were added to each well of 96 well plate along with 20 IU/ml IL-2. Cells were incubated at 37° C. for 24 hrs either with B16 or control EL4 cells. After culture, the plate was washed and then followed by incubation with 5 mg/ml biotinylated IFNγ antibody (clone XMG1.2, PharMingen) in 50 ml in PBS at 4° C. overnight. After washing six times, 1.25 mg/ml avidin-alkalinephosphatase (Sigma, St. Louis, Mo.) in 50 ml of PBS were added and incubated for 2 hrs at room temperature. After washing, spots were developed by adding 50 ml of 5-bromo-4-chloro-3-indolyl phosphate/nitroblue tetrazolium solution (Boehringer Mannheim, Indianapolis, Ind.) and incubated at room temperature for 1 hr. The spots were counted using a dissecting microscope.

Immunization and Tumor Protection Assay

One hundred micrograms of purified hTRPx3 was emulsified in 100 □l complete Freund's adjuvant (CFA) for each injection. C57BL/6 mice were vaccinated subcutaneously every 10 days for five times and bled one week after boosting. To compare different adjuvant, some mice were immunized with hTRPx3/BCG or hTRPx3/GMCSF intradermally. Unless stated, all results were from mice vaccinated with hTRPx3/CFA. For melanoma protection assay, mice were injected intravenously through the tail vein with 5×10⁴ B16BL6 cells 10 days after last immunization. Mice were sacrificed and lungs were removed 20 days after B16 melanoma challenge. The surface lung metastases were scored and counted under a dissecting microscope. Statistical analysis of surface lung metastases was performed using student t test.

### Example 1

The present example demonstrates construction and production of a tri-hybrid melanoma antigen, recombinant hTRPx3 protein.

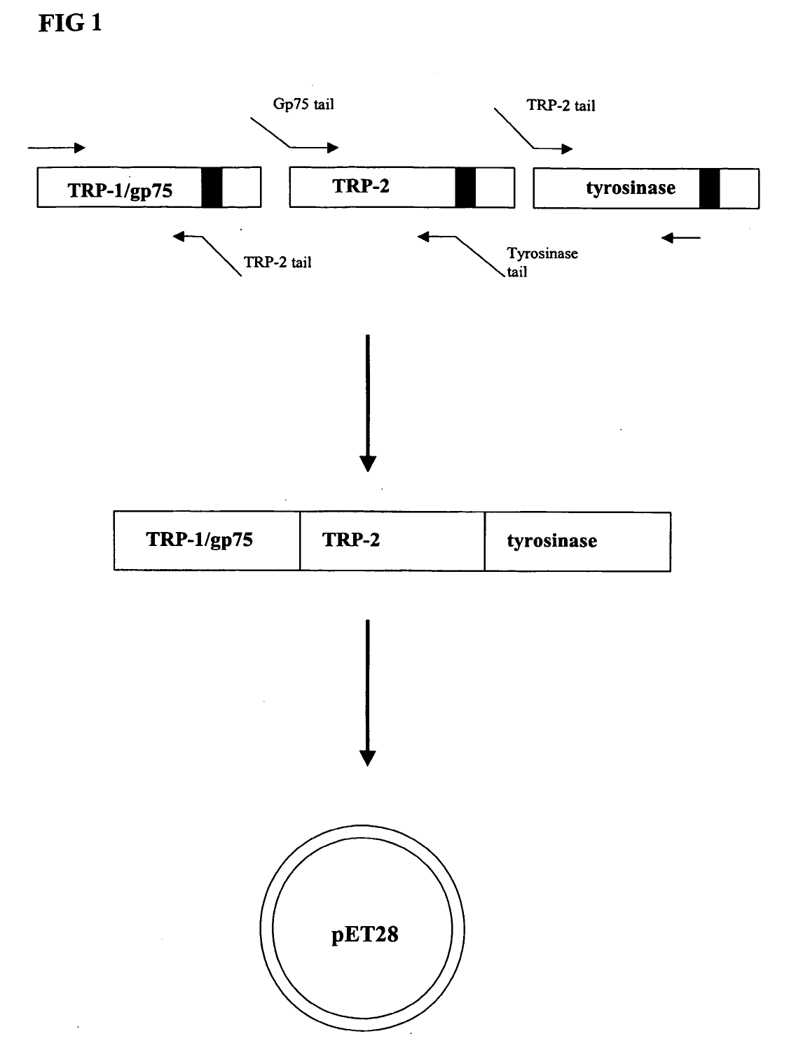

To generate a chimeric hTRPx3 molecule, a DNA fragment encoding human TRP-1, TRP-2, and tyrosinase were made using PCR primer pairs as shown above (FIG. 1). Each PCR resulted a 1.4 kb fragment (PCR product one). The resulting three PCR products were then mixed and underwent a 10-cycle PCR reaction (PCR product two). Finally, PCR product two was amplified with primers htyr-1 and htyr-6 (PCR product three). PCR product three was about 4.1 kb and was digested with EcoR I and Xho I before cloning into pET28 (Novogen, bacteria expression vector). The final product was confirmed by sequencing. The coding sequence of the cloned tri-hybrid nucleotide sequences was determined by standard techniques and is given by SEQ ID NO:7. The amino acid sequence of the translation product is given by SEQ ID NO:8.

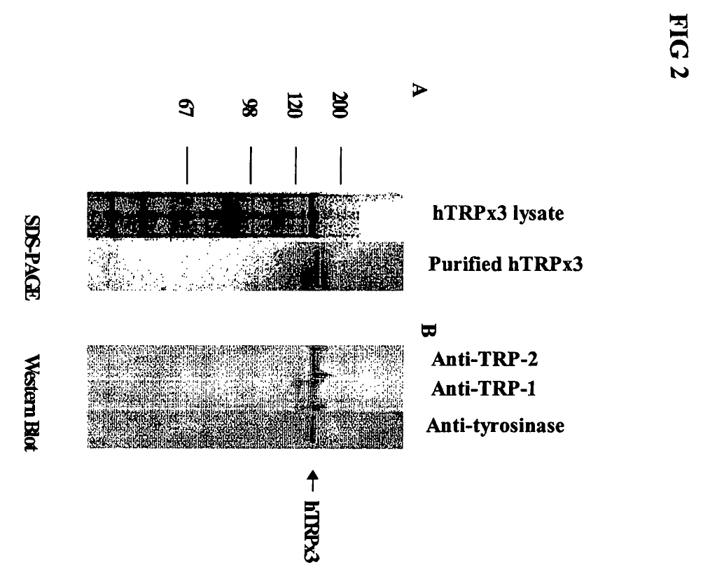

Induction of BL21 E. coli cells containing plasmids encoding human hTRPx3 genes resulted in expression of 155-kDa-fusion proteins in insoluble forms (FIG. 2A). The supernatant fraction (i.e. the soluble protein) from cell lysates showed little amounts of hTRPx3 proteins in soluble form. No inhibition of cell growth during the induction was observed, indicating that human TRPx3 was not toxic when expressed in the fusion proteins. The level of expressed proteins could be increased if cells were induced at 37° C. due to the formation of inclusion bodies. The level of the expressed TRP1 protein was about 5 mg/L. The expressed hTRPx3 proteins were conformed by Western analysis using anti-sera specific for either TRP-1, or TRP-2 or tyrosinase (FIG. 2B).

### Example 2

The present example demonstrates induced of a humoral immune response following immunization with a tri-hybrid melanoma antigen, hTRPx3 protein.

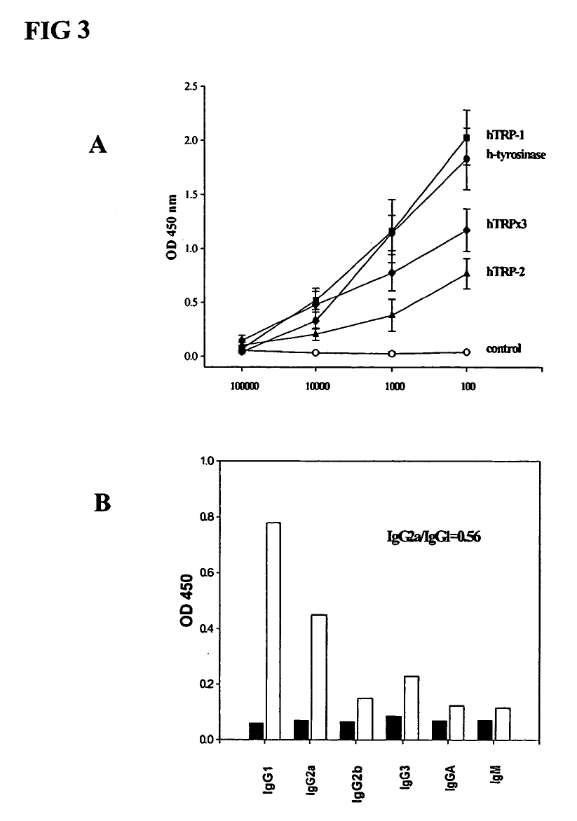

Protein immunization with hTRPx3 protein was performed on 10 mice per group. One week after last boost, serum samples were tested for their ability to react with the purified individual antigens on ELISA plates (FIG. 3A). A majority of immunized mice developed antibody responses against individual proteins and hTRPx3 (27/30).

To determine the subtypes of antibodies involved in these responses, antibody subtyping was performed on the ELISA plates (FIG. 3B). Briefly, the immune plates were coded with recombinant hTRPx3 and then incubated with sera from immunized mice. The bound antibodies were determined by second antibodies specific for mouse immunoglobulin IgG₁, IgG₂ₐ, IgG_{2b}, IgG₃, and IgM. Antibodies against hTRPx3 were IgG subclass; predominantly IgG₁ and IgG₂ₐ (IgG₂ₐ/IgG₁=0.56) suggesting that Th1 and Th2 responses were induced.

### Example 3

The present example demonstrates that immunization with a tri-hybrid melanoma antigen, hTRPx3 protein, induced a T cell immune response.

To determine if T cells specific for individual antigens were induced in mice, splenocytes from hTRPx3 immunized mice were incubated with hTRP-1, hTRP-2, and tyrosinase protein respectively. The IFNγ released by the T cells in the supernatant was determined by standard IFNγ kits.

The splenocytes from hTRPx3 immunized mice were found to release significantly higher amounts of IFNγ as compared to control mice following stimulation by antigens (8, 4, and 4-fold increase in IFNγ release upon hTRP-1, hTRP-2, and h-tyrosinase stimulation, respectively). Splenocytes from saline control mice also released IFNγ when stimulated with individual antigens, suggesting these recombinant proteins can have induced some T cell activation in vitro.

B16 melanoma cells express all tyrosinase family members and MHC class I molecules. T cells specific for hTRPx3 should also react with syngeneic B16 cells. To confirm this, ELISPOT assays were conducted. Briefly, different concentrations of fresh isolated spleen cells from each vaccinated mice group were added to the well of ELISPOT plate. Cells were incubated either with B16 or EL4 control cells. The IFNγ released were captured by mAb to mouse IFNγ on ELISPOT plate.

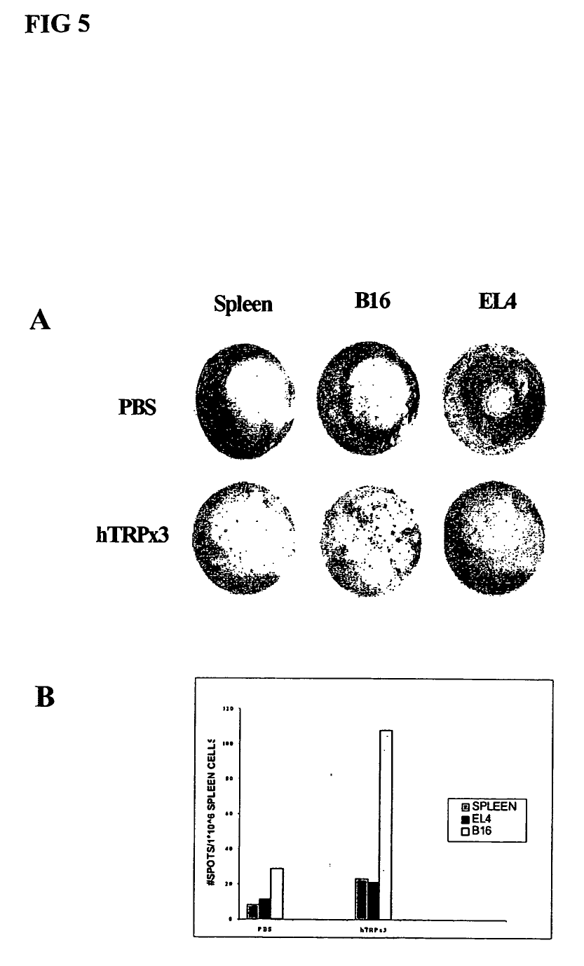

As shown in FIG. 5, splenocytes from hTRPx3 immunized mice release IFNγ upon B16 cell stimulation. Since B16 cell did not express MHC class II molecule, it is highly possible that CD8 T cells were responsible for these IFNγ spots.

### Example 4

The present example demonstrates that immunization with a tri-hybrid melanoma antigen, hTPRx3 protein, was useful in treating tumors in mammals.

To determine the effects of autoimmunity to hTRPx3 on melanoma in vivo, a syngeneic mouse model was used. Ten days after the last booster, immunized C57BL/6 mice were injected intravenously (i.v.) with B16BL6 melanoma cells, which is a spontaneously occurring melanoma from C57BL/6 mouse.

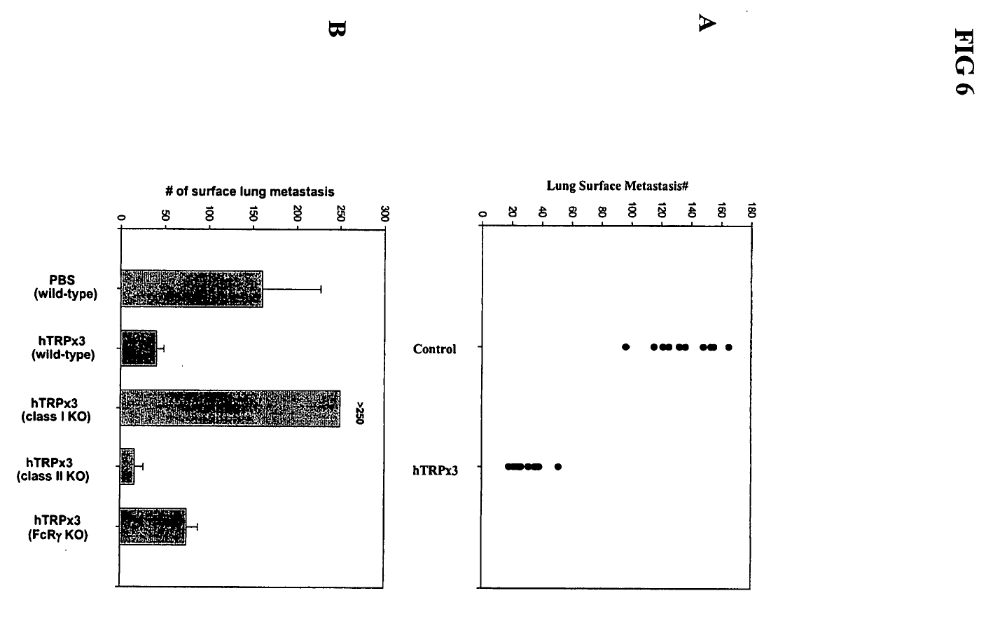

Mice immunized with recombinant hTRPx3 were significantly protected from lung metastases of B16BL16 melanoma (FIG. 6A). In comparison with control mice, hTRPx3 protein immunized mice had a significant less lung metastases, with 80% (p=0.001) fewer lung nodules.

To determine the mechanisms of antitumor activity, tumor protection was evaluated following hTRPx3 immunization in MHC class I, class II, and FcR knock-out mice (FIG. 6B). Mice with deficiency in MHC class I have lost tumor protection. Deficiency in MHC class II molecules has no effect on antitumor activity in mice, suggesting that CD8 cells can play a key role in hTRPx3 mediated antitumor activity.

hTRPx3 immunization was further tested using different adjuvants. Both BCG and GM-CSF have been reported as Th1 driven adjuvants (see, e.g., Disis et al., Blood, 88(1):202-10 (July 1996); Kumar et al., Immunology, 97(3):515-21 (July 1999)). Mice vaccinated with hTRPx3/BCG were protected from melanoma challenge similar to mice immunized with hTRPx3/CFA. Mice vaccinated with hTRPx3/GM-CSF were not protected from tumor challenge.

We claim:

1. An isolated DNA encoding a tri-hybrid melanoma antigen comprising tyrosinase or a fragment thereof, tyrosinase-related protein 1 (TRP-1) or a fragment thereof, and tyrosinase-related protein 2 (TRP-2) or a fragment thereof.

2. The isolated DNA of claim 1, wherein the tri-hybrid melanoma antigen comprises SEQ ID NO:7 or a fragment thereof, SEQ ID NO:9 or a fragment thereof, or SEQ ID NO:11 or a fragment thereof.

3. A cloning or expression vector comprising the DNA of claim 1 or 2.

4. A host cell comprising the cloning or expression vector of claim 3.

5. An isolated tri-hybrid melanoma antigen comprising tyrosinase or a fragment thereof, tyrosinase-related protein 1 (TRP-1) or a fragment thereof, and tyrosinase-related protein 2 (TRP-2) or a fragment thereof.

6. The isolated tri-hybrid melanoma antigen of claim 5, wherein the tri-hybrid melanoma antigen comprises SEQ ID NO:8 or a fragment thereof, SEQ ID NO:10 or a fragment thereof, or SEQ ID NO:12 or a fragment thereof.

7. A composition comprising the isolated DNA of claim 1 or 2 and a pharmaceutically acceptable carrier.

8. A composition comprising the isolated tri-hybrid melanoma antigen of claim 5 or 6 and a pharmaceutically acceptable carrier.

9. A composition for inhibiting melanosomal activity in an animal comprising a tri-hybrid melanoma antigen or a fragment thereof and a pharmaceutically acceptable carrier.

10. A composition for inhibiting tumor growth in an animal comprising a tri-hybrid melanoma antigen or a fragment thereof and a pharmaceutically acceptable carrier.

11. A composition for vaccination comprising a tri-hybrid melanoma antigen or a fragment thereof and a pharmaceutically acceptable carrier.

12. The composition of any of claims 7-11, wherein the tri-hybrid melanoma antigen or a fragment thereof comprises tyrosinase or a fragment thereof, tyrosinase-related protein 1 (TRP-1) or a fragment thereof, and tyrosinase-related protein 2 (TRP-2) or a fragment thereof.

13. The composition of any of claims 7-12, wherein the tri-hybrid melanoma antigen or a fragment thereof comprises SEQ ID NO:8 or a fragment thereof, SEQ ID NO:10 or a fragment thereof, or SEQ ID NO:12 or a fragment thereof.

14. A method of eliciting an immune response against a melanosomal antigen in an animal comprising administering to the animal an effective amount of a tri-hybrid melanoma antigen or a fragment thereof.

15. A method of treating a tumor in an animal comprising administering to the animal an effective amount of a tri-hybrid melanoma antigen or a fragment thereof.

16. A method of vaccination in an animal comprising administering to the animal an effective amount of a tri-hybrid melanoma antigen or a fragment thereof.

17. The method of any of claims 14-16, wherein the tri-hybrid melanoma antigen or a fragment thereof comprises tyrosinase or a fragment thereof, tyrosinase-related protein 1 (TRP-1) or a fragment thereof, and tyrosinase-related protein 2 (TRP-2) or a fragment thereof.

18. The method of any of claims 14-17, wherein the tri-hybrid melanoma antigen or a fragment thereof comprises SEQ ID NO:8 or a fragment thereof, SEQ ID NO:10 or a fragment thereof, or SEQ ID NO:12 or a fragment thereof.

19. The method of any of claims 14-18, wherein the method induces production of an antibody that specifically binds a melanosomal antigen.

20. The method of any of claims 14-19, wherein the method induces production of IFNγ.

21. The method of any of claims 14-20, wherein the method induces production of CD8 T cells.

22. The method of any of claims 14-21, wherein the method induces production of cytotoxic lymphocytes specific for a melanosomal antigen.

23. The method of any of claims 14-22, wherein the method is used to treat a condition associated with excess melanosomal antigen activity in the animal.

24. The method of claim 23, wherein the condition is a tumor.

25. The method of claim 24, wherein the tumor is a malignant tumor.

26. The method of claim 25, wherein the malignant tumor is a carcinoma, sarcoma, leukemia, or a lymphoma.

27. The method of any of claims 24-26, wherein the method prevents metastasis of the tumor.

28. The method of claim 23, wherein the condition is a pre-malignant lesion.

29. The method of claim 28, wherein the pre-malignant lesion is a adenoma or dysplastic lesion.

30. The method of any of claims 14-29, wherein the tri-hybrid melanoma antigen is administered in combination with an adjuvant.

31. The method of any of claims 14-30, wherein the animal is a mammal.

32. The method of claim 31, wherein the animal is a human.
